(19) United States
(12) Patent Application Publication
(10) Pub. No.: US 2026/0250259 A1
(43) Pub. Date: Aug. 27, 2026

(54) PRODUCING 2,5-FURANDICARBOXYLIC ACID WITH RECOVERY OF ACID

(71) Applicant: Furanix Technologies B.V., Amsterdam (NL)

(72) Inventors: Jeffrey John Kolstad, Amsterdam (NL); Jan Hendrik Blank, Amsterdam (NL); Gerardus Johannes Maria Gruter, Amsterdam (NL)

(21) Appl. No.: 18/871,235

(22) PCT Filed: Jun. 13, 2023
(86) PCT No.: PCT/EP2023/065711

§ 371 Date: Dec. 3, 2024

(30) Foreign Application Priority Data
Jun. 14, 2022 (EP) 22178817.7

(51) Int. Cl. C07D 307/68

(52) U.S. Cl. CPC C07D 307/68

(57) ABSTRACT

Process for producing 2,5-furandicarboxylic acid comprising the steps of: a) oxidizing feed comprising 5-methylfurfural using an oxidizing gas at a temperature in the range of 150 to 210° C. to obtain a crude carboxylic acid composition comprising 2,5-furandicarboxylic acid, and a catalyst system comprising cobalt, manganese and bromine; b) separating solid 2,5-furandicarboxylic acid from the crude carboxylic acid composition in a solid-liquid separation zone to obtain a solid cake and mother liquor; and c) treating the cake with solvent selected from the group consisting of water and mono- and/or dicarboxylic acids containing of from 1 to 3 carbon atoms to obtain a treated cake and solvent having increased organic acid content which solvent having increased organic acid content comprises 5-bromo-2-furancarboxylic acid and wherein at least part of the solvent having increased organic acid content is added to step a).

### TECHNICAL FIELD

The present invention relates to a process for producing 2,5-furandicarboxylic acid, specifically a process for producing 2,5-furandicarboxylic acid using 5-methylfurfural as starting material.

### BACKGROUND

2,5-Furandicarboxylic acid (FDCA) is known in the art to be a highly promising building block for replacing petroleum-based monomers in the production of high performance polymers. In recent years, 2,5-furandicarboxylic acid and the novel plant-based polyester polyethylenefuranoate (PEF), a completely recyclable plastic with superior performance properties compared to today's widely used petroleum-based plastics, have attracted a lot of attention. These materials could provide a significant contribution to reducing the dependence on petroleum-based polymers and plastics, while at the same time allowing for a more sustainable management of global resources. Comprehensive research was conducted in the field to arrive at a technology for producing 2,5-furandicarboxylic acid and PEF in a commercially viable way.

2,5-Furandicarboxylic acid is typically obtained by oxidation of molecules having furan moieties, e.g. 5-hydroxymethylfurfural (5-HMF) as well as the corresponding esters and ethers, e.g. 5-alkoxymethylfurfural, and similar starting materials, that are typically obtained from plant-based sugars, e.g. by sugar dehydration. A broad variety of oxidation processes is known from the prior art such as enzymatic and metal catalysed processes, either heterogeneous or homogeneous. WO 2014/014981 and WO 2011/043661 describe processes using catalyst systems comprising cobalt, manganese and bromine to oxidize compounds having a furan moiety to 2,5-furandicarboxylic acid using oxygen or air as an oxidizing agent.

The purity of crude 2,5-furandicarboxylic acid product is oftentimes not sufficient for use in the manufacture of polymers having desirable properties. The product obtained by oxidation can be colored. Color is disadvantageous in that it indicates the presence of impurities. In order to objectively determine color, absorbance of a solution is to be measured at a particular wavelength such as 400 nm. Impurities which lead to color are often difficult to specifically identify making their removal difficult.

Processes have been developed for further purifying crude oxidation products. Exemplary purification processes are disclosed in WO 2014/014981 and WO 2016/195499.

It can be advantageous to be able to use 5-methylfurfural (5-MF) for oxidation into 2,5-furandicarboxylic acid as the availability of 5-methylfurfural may increase in the future. Processes which produce 5-hydroxymethylfurfural (5-HMF), as well as corresponding esters or ethers, frequently use sugar as feedstock such as glucose or especially fructose. In contrast, processes to make 5-methylfurfural can start from biomass or cellulosic materials which do not compete with the food chain and which may have reduced environmental impact. Furthermore, it could be expected that less by-products are formed in the conversion of 5-methylfurfural due to methyl group which tends to be less reactive.

While oxidation of compounds such as 5-hydroxymethylfurfural and ethers thereof has been extensively studied, less is known about oxidation of 5-methylfurfural. Soviet Union Inventor's Certificate 441877 describes the conversion of 5-methylfurfural into 2,5-furandicarboxylic acid at low yield and unknown purity. WO2011043661 mentions 5-methylfurfural as a possible feed for oxidation. Examples 3a and 3b using 5-methylfurfural gave a lower yield of 2,5-furandicarboxylic acid than Examples 1d and 1 h obtained from 5-hydroxymethylfurfural, both at 100% conversion. Therefore, the product obtained from 5-methylfurfural contained more compounds other than the free diacid, i.e. 2,5-furandicarboxylic acid, than the product obtained from 5-hydroxymethylfurfural.

In addition, it was also found that crude 2,5-furandicarboxylic acid obtained from 5-methylfurfural can suffer from incorporation of catalyst metals, more specifically manganese and/or cobalt, into the product cake. This not only contaminates the product but also withdraws valuable catalyst components from the system that could otherwise be reused or recycled.

### DISCLOSURE OF THE INVENTION

It was an objective to improve the purity of the crude carboxylic acid obtained by oxidation of 5-methylfurfural. A further objective was to improve, i.e. reduce, the absorbance of the crude carboxylic acid obtained.

It was found that crude 2,5-furandicarboxylic acid which was obtained by 5-methylfurfural oxidation contained a surprising impurity namely 5-bromo-2-furoic acid also referred to as 5-bromo-2-furancarboxylic acid (Br-FCA).

Surprisingly, it now has been found that the amount of Br-FCA can be reduced by contacting the crude 2,5-furandicarboxylic acid with solvent selected from the group consisting of water and mono- and/or dicarboxylic acids containing of from 1 to 3 carbon atoms. Furthermore, adding Br-FCA containing solvent to step a) can improve oxidation of 5-methylfurfural.

The invention relates to a process for producing 2,5-furandicarboxylic acid, comprising the steps of: a) oxidizing feed comprising 5-methylfurfural using an oxidizing gas at a temperature in the range of 150 to 210° C. to obtain a crude carboxylic acid composition comprising 2,5-furandicarboxylic acid, and a catalyst system comprising cobalt, manganese and bromine; b) separating solid 2,5-furandicarboxylic acid from the crude carboxylic acid composition in a solid-liquid separation zone to obtain a solid cake and mother liquor; and c) treating the cake with solvent selected from the group consisting of water and mono- and/or dicarboxylic acids containing of from 1 to 3 carbon atoms to obtain a treated cake and solvent having increased organic acid content which solvent having increased organic acid content comprises 5-bromo-2-furancarboxylic acid and wherein at least part of the solvent having increased organic acid content is added to step a).

### MODES FOR CARRYING OUT THE INVENTION

The process is aimed at producing 2,5-furandicarboxylic acid. The product obtained can contain further compounds besides 2,5-furandicarboxylic acid especially if the final product obtained is the treated cake produced in step c). The product obtained after further treatments such as hydrogenation will tend to contain a lower amount of further compounds besides 2,5-furandicarboxylic acid.

Step a) comprises oxidation of a feed comprising 5-methylfurfural. The feed comprises at least 5-methylfurfural optionally in combination with further compounds. The feed preferably comprises at least 80% by weight of 5-methylfurfural, more preferably at least 90% by weight of 5-methylfurfural. The feed preferably consists of 5-methylfurfural. Besides the feed, the mixture present in step a) comprises oxidizing gas, acetic acid and a catalyst system. After the reaction has started, the mixture present in step a) tends to further contain water produced by oxidation of 5-methylfurfural.

The treated cake preferably contains at most 5000 parts per million by weight (ppmw) of 5-bromo-2-furoic acid (Br-FCA), based on weight amount of dry cake, more preferably at most 2000 ppmw, more preferably at most 1000 ppmw, most preferably at most 500 ppmw.

Step a) preferably is carried out at a temperature in the range of 150 to 210° C., preferably a temperature of 160 to 190° C., more preferably a temperature in the range of from 165 to 180° C. Preferably, the pressure in step a) is in the range of 700 to 2000 kPa. These parameters were found to be preferred for obtaining 2,5-furandicarboxylic acid of good purity in good yields while at the same time enabling the reactors to be run such that the substantial heat generated by oxidation is removed by vaporization of a portion of the solvent. This is known in the art as adiabatic operation.

The catalyst system comprises cobalt, manganese and bromine either as the element or as a derivative thereof. The catalyst system preferably has a weight ratio of cobalt to manganese in the catalyst system of 10 or higher, preferably 15 or higher, and/or a weight ratio of bromine to the combined weight of cobalt and manganese in the catalyst system of 1 or higher, preferably 1.5 or higher, most preferably 2 or higher, wherein the value is preferably less than 4.0, more preferably less than 3.5. If the catalyst system comprises other metals besides cobalt and manganese in an amount of 5% by weight or more, it is preferred that the above ratios are achieved for the weight ratio of bromine to the combined weight of all metals in the catalyst system. The metals preferably are added as salts which are soluble in the reaction mixture. Typically, the amount of cobalt is selected in the range of 500 to 6000 ppm by weight, based on the weight of the feed, acetic acid and catalyst system. The amount of manganese typically is in the range from 20 to 6000 ppm by weight, based on the weight of the feed, acetic acid and catalyst system Typically, the bromine concentration would be from 30 to 8000, preferably 50 to 4500 ppm by weight of bromine, based on weight of the the feed, acetic acid and catalyst system. Alternatively, the bromine content is from 3000 to 8000 ppm by weight.

The oxidizing gas can be any gas known to be suitable by the person skilled in the art. Preferably, the oxidizing gas comprises molecular oxygen. Most preferably, the oxidizing gas is air.

The reactor for carrying out the oxidation can be any typical oxidation reactor that is known in the art.

A post-oxidation step has been found to be preferred especially when employed at high temperature. Most preferred is a process wherein a post oxidation step a1) is applied after step a) at a temperature of at a temperature in the range of 150 to 210° C., more specifically of 160 to 210° C.

In step b) solid 2,5-furandicarboxylic acid is separated from the crude carboxylic acid composition in a solid-liquid separation zone to obtain a solid cake and mother liquor. This means that solid containing 2,5-furandicarboxylic acid is separated from the crude carboxylic acid composition. Not all of the 2,5-furandicarboxylic acid generally will be removed from the crude carboxylic acid composition while generally not all of the solid cake which is separated will be 2,5-furandicarboxylic acid.

Preferably, at least 50% by weight with respect to the weight of the cake will be 2,5-furandicarboxylic acid, more preferably at least 70% by weight, more preferably at least 80% by weight, more preferably at least 90% by weight, most preferably at least 95% by weight. Other compounds which can be present as part of the cake are derivatives of 2,5-furandicarboxylic acid such as methyl ester of 2,5-furandicarboxylic acid, 5-hydroxymethyl-furan-2-carboxylic acid (HMFCA), 2-carboxy-5-(formyl) furan (FFCA), 5-bromo-2-furoic acid (Br-FCA) and bis-carbonyl-furoic acid, also referred to as 5,5′-carbonyl-bis-furan-2-carboxylic acid, (BCFCA).

The separation of step b) can be carried out in any way known to the person skilled in the art. Preferred is a process wherein the solid-liquid separation zone comprises a filter or centrifuge, preferably a filter, more preferably a rotary pressure filter.

In continuous operation, at least a portion, preferably at least 60% by weight, more preferably at least 80% by weight, of the mother liquor preferably is routed from the solid-liquid separation zone to the oxidation reactor as recycled mother liquor stream.

In step c), the cake is treated with solvent selected from the group consisting of water and mono- and/or dicarboxylic acids containing of from 1 to 3 carbon atoms to obtain a treated cake and solvent having an increased organic acid content. It will be clear that dicarboxylic acids will contain at least 2 carbon atoms.

The person skilled in the art will know how to treat the cake to dissolve the 5-bromo-2-furoic acid (Br-FCA). In a preferred embodiment, the cake is treated by mixing the cake with the solvent. The volume of solvent preferably substantially exceeds the volume of the cake. Most preferably, the cake and solvent are mixed thoroughly after which the treated cake is separated from the solvent in a solid-separation zone, preferably by filtering. In an especially preferred embodiment the amount of solvent and the temperature are chosen in combination with each other, such that a substantial portion of the cake is dissolved, but not all of the cake. The portion of the cake which is dissolved is preferably in the range of from 10 to 80% by weight (wt %), and more preferably of from 20 to 60 wt %, and most preferably in the range of from 30 to 50 wt % based on amount of dry cake. Suitable contact time at temperature are from preferably from 15 minutes to 4 hours, more preferably from 30 minutes to 2 hours. It is preferred that the percentage of dissolved cake is in the given range when averaged over the duration of the treatment.

Preferably, the solvent comprises water and/or acetic acid. More preferably, the solvent is selected from the group consisting of water, acetic acid and mixtures thereof.

Preferred is a process according to the invention, wherein the treated cake comprises 2,5-furandicarboxylic acid in an amount greater than 95%, preferably greater than 98%, by weight with respect to the weight of the dry solids.

At least part of the solvent having increased organic acid content obtained in step c) is added to step a). The solvent having increased acid content comprises 5-bromo-2-furancarboxylic acid. It will be clear to the person skilled in the art that all or part of the solvent having increased organic acid content can be added to step a). It can be preferred that a bleed stream prevents building up contaminants. Preferably, the solvent having increased organic acid content is added to step a).

In a preferred embodiment, the process further comprises d) contacting treated cake with polar solvent to obtain a solution; e) contacting the solution with hydrogen in the presence of a hydrogenation catalyst at hydrogenation conditions yielding a hydrogenated solution; and f) separating purified 2,5-furandicarboxylic acid from the hydrogenated solution, preferably separating by crystallization. Suitable process conditions are for example described in WO2016/195490. Preferred process conditions comprise contacting with hydrogen at a temperature in the range of 150 to 200° C. and a contact time with the hydrogenation catalyst in the range of 5 seconds to 15 min. Preferably, the polar solvent is selected from the group consisting of water, acetic acid and mixtures thereof.

It will be clear to the person skilled in the art that preferably all solution is subjected to step e) although it is possible to use part of the solution only.

Hereinafter, the invention is described in more detail using experiments.

### Example 1

The oxidation reactor is a 600 ml stirred pressure vessel, with two impellors. The reactor is pre-charged with a mixture having a total weight of 310 grams. The mixture comprises catalyst components provided as cobalt (II) acetate tetrahydrate, manganese (II) acetate tetrahydrate, and HBr as 48% by weight (wt %) in water. The amounts of the catalyst components are such as to yield a mixture which contained 3300 ppm Co, 188 ppm Mn and 7000 ppm Br. Water is added in an amount to result in 5 wt % of the total mixture, after accounting for the water introduced as part of the catalyst components. The balance is acetic acid.

The oxidation reactor is purged, pressurized, and heated to the desired operating temperature with stirring at 2000 rpm. The feed of Experiment 1 was 5-methoxymethylfurfural (MMF), the feed for Experiments 2, 3 and 5 was 5-methyl furfural (5-MF), the feed for Experiment 4 was 5-hydroxymethyl-2-furaldehyde (5-HMF) and the feed for Experiment 6 was a mixture of 5-methyl furfural (MF) and 5-methylmethoxy furfural (MMF) (weight ratio 70/30).

The process is started with a typical feed rate 8.3 mmol/minute. This feed rate was continued for 60 minutes (total feed 500 mmol) in the first set of experiments (Experiments 1, 2 and 3) and for 30 minutes (total feed 250 mmol) in the second set of experiments (Experiments 4, 5 and 6).

The oxidation reactor was purged, pressurized and heated to the desired operating temperature with stirring at 2000 rpm. A flow rate of lean air (8% oxygen) is started at a typical flow rate of 10 normal L/minute. The reaction typically begins within 3 minutes, noticed by a sharp decrease in oxygen in the outlet and an increase in CO and CO₂. During the reaction heat is generated, and a vapor stream is taken overhead and condensed. This vapor stream comprises mainly of acetic acid and water. The amount of solvent captured in the overhead is continuously monitored, and made up in the oxidation reactor with a fresh flow of solvent to the reactor.

The typical operating pressure was 12 to 14 barg at 160° C. oxidation temperature.

At the end of the desired feed period, the feed of oxidizable compound is stopped, and the contents of the oxidation reaction is subjected to a period of post-oxidation.

The oxidation was followed by post-oxidation. Post-oxidation was conducted by stopping the flow of lean air for 1 minute and then re-establishing lean air flow at 4 NI/min for 20 minutes while maintaining the reaction temperature at 160° C.

Solids were separated by filtration and the cake obtained was washed twice with 1 part solvent (95 acetic acid to 5 parts water, by weight) to 1 part estimated dry cake weight each time.

The cake absorbance was measured by mixing 300 mg of crude 2,5-furandicarboxylic acid with 10 ml of dimethyl sulfoxide (DMSO). To ensure complete dissolution, the solution was allowed to stand for 4 hours. The absorbance of this solution was measured in a 1 cm cell in a UV/VIS photo-spectrometer against a DMSO standard using a wavelength of 400 nm.

The amount of cobalt and manganese were determined by inductively coupled plasma or ICP analysis.

The amount of 5-bromo-2-furoic acid (Br-FCA) was determined by ultra high performance liquid chromatography (UPLC) gradient elution on a Waters C18 column. Eluent A was water containing 0.2 vol % trifluoroacetic acid and eluent B was a 50/50 by volume mixture of methanol and acetonitrile.

The results are shown in the below table.

TABLE 1
Oxidation of feed

| | | Amount of | Br-FCA | | | UV absor- |
|---|---|---|---|---|---|---|
| | | feed | solid | Co | Mn | bance |
| # | Feed | (mmol) | (ppm) | (ppm) | (ppm) | 400 nm |
| Exp. 1 | MMF | 500 | 41 | 2240 | 460 | 0.020 |
| Exp. 2 | MF | 500 | 1285 | 2120 | 360 | 0.110 |
| Exp. 3 | MF | 500 | 1373 | 2490 | 400 | 0.259 |
| Exp. 4 | HMF | 250 | 368 | 4826 | 542 | |
| Exp. 5 | MF | 250 | 3076 | 1970 | 430 | 0.053 |
| Exp. 6 | 70 MF/ | 250 | 1733 | 425 | 176 | 0.014 |
| | 30 MMF | | | | | |

Table 1 shows that a feed comprising 5-methylfurfural or a mixture of 5-methylfurfural and alkoxymethyl-2,5-furfural resulted in solid 2,5-furandicarboxylic acid having an increased content of 5-bromo-2-furoic acid. Recovering 5-bromo-2-furoic acid from the cake allows to add it to step a) thereby obtaining a cake having improved properties.

1. A process for producing 2,5-furandicarboxylic acid, comprising the steps of:
a) oxidizing feed comprising 5-methylfurfural using an oxidizing gas at a temperature in the range of 150 to 210° C. to obtain a crude carboxylic acid composition comprising 2,5-furandicarboxylic acid, and a catalyst system comprising cobalt, manganese and bromine;
b) separating solid 2,5-furandicarboxylic acid from the crude carboxylic acid composition in a solid-liquid separation zone to obtain a solid cake and mother liquor; and
c) treating the cake with solvent selected from the group consisting of water and mono- and/or dicarboxylic acids containing of from 1 to 3 carbon atoms to obtain a treated cake and solvent having increased organic acid content which solvent having increased organic acid content comprises 5-bromo-2-furancarboxylic acid, and wherein at least part of the solvent having increased organic acid content is added to step a).

2. The process according to claim 1 wherein the solvent comprises water and/or acetic acid.

3. The process according to claim 1 or 2 wherein the treated cake contains less than 1000 parts per million by weight of 5-bromo-2-furoic acid based on weight amount of dry cake.

4. The process according to claim 1 wherein the cake is treated by mixing the cake with the solvent and wherein the portion of the cake which is dissolved is in the range of from 10 to 80% by weight based on amount of dry cake.

5. The process according to claim 1 wherein the solvent is selected from the group consisting of water, acetic acid and mixtures thereof.

6. The process according to claim 1, which process further comprises
d) contacting treated cake with polar solvent to obtain a solution;
e) contacting the solution with hydrogen in the presence of a hydrogenation catalyst at hydrogenation conditions yielding a hydrogenated solution; and
f) separating purified 2,5-furandicarboxylic acid from the hydrogenated solution.
